(19) United States
(12) Patent Application Publication
(10) Pub. No.: US 2008/0208177 A1
(43) Pub. Date: Aug. 28, 2008

(54) Opthalmological Device

(76) Inventors: Michael Mrochen, Eglisau (CH); Theo Seiler, Zurich (CH)

Correspondence Address: STRAUB & POKOTYLO, 788 Shrewsbury Avenue, TINTON FALLS, NJ, 07724, US

(21) Appl. No.: 10/588,175

(22) PCT Filed: Feb. 3, 2005
(86) PCT No.: PCT/EP2005/001083

§ 371 Date: Apr. 25, 2008

(30) Foreign Application Priority Data
Feb. 3, 2004 (EP) 04002326.9

(51) Int. Cl. A61F 9/01; A61B 18/20

(52) U.S. Cl. 606/5

(57) ABSTRACT

A device for correcting defective vision or corneal disease of an eye combines a device (16) for deforming the cornea of the eye and a device (18, 20) for hardening the cornea.

The invention relates to a device for correcting defective vision or corneal disease of an eye, as well as to instruments for using such a device.

So-called keratoconus is a disease which entails softening of the eye's cornea and, because of this softening, corneal bulging due to the internal pressure of the eye. It is clear that such bulging leads to perturbation of the imaging properties of the eye. A conservative therapy of keratoconus involves hardening the cornea. This is described, for example, in the following publications: E. Spörl, J. Schreiber, K. Hellmund, T. Seiler and P. Knuschke in DER OPHTALMOLOGE 3-2000, pp. 203-206; E. Spörl, T. Seiler in JOURNAL OF REFRACTIVE SURGERY, Vol. 15, 1999, pp. 711-713; G. Wollensak, E. Spoerl, T. Seiler, AMERICAN JOURNAL OF OPHTHALMOLOGY, May 2003, pp. 620-627. Expressed concisely, according to this prior art for the conservative therapy of keratoconus the epithelium of the cornea is first removed and then a photosensitiser (e.g. riboflavin) is applied onto the exposed cornea. This photosensitiser then penetrates through the entire cornea and also reaches into the anterior chamber of the eye. The eye is then irradiated with selected electromagnetic radiation (for example UVA or UV) so as to induce biochemical and biomechanical processes (for example cross-linking) which lead to hardening of the cornea. As one of the body's own products, the photosensitiser is subsequently broken down within a relatively short time without leaving a residue. The mechanical hardening of the tissue which is achieved more or less prevents the said undesired bulging of the cornea.

So-called orthokeratology is another known correction for defective vision of the eye. In this conservative therapy the patient wears a special contact lens (for example over night) which deforms the cornea in the desired way. If the deforming contact lens is left on the eye for a prolonged period of time, for example several hours, then the deforming effect can persist over fairly long periods of time after the contact lens is removed, and thus lead to a reduction of the defective vision. This corrective effect is not stable, however, particularly in patients with weak mechanical properties of the cornea. The variation in the refractive properties of the eye which occurs in this method may also be perceived as disturbing by patients.

It is an object of the present invention to provide a device and a method with which the aforementioned imaging errors and weaknesses of the eye can be treated more effectively.

To this end, the invention provides a device in which an instrument for deforming the cornea and an instrument for hardening the cornea are combined.

The deformation and hardening of the cornea may take place simultaneously or with a time delay or time overlap. In general, the hardening is carried out when the deformation is present.

The instrument for deforming the cornea preferably comprises a shaped body which can be placed on the eye, i.e. for example a contact lens known per se or the like. For the device according to the invention, however, the shaped body need not necessarily be configured like a contact lens which optimally improves the sight of the eye; rather, the shaped body may be optimised by taking into account the corneal hardening which will be described in detail below.

The aforementioned instrument for deforming the cornea preferably comprises a shaped body which is suitable for being applied onto the cornea so is to create a negative pressure (vacuum) between the cornea and the shaped body, by which the cornea is deformed i.e. fits tightly onto the surface of the shaped body in the entire desired region.

The hardening of the cornea, which has been brought into a desired shape, is carried out with a device according to the invention by at least one radiation source for irradiating the cornea, preferably with the radiation homogeneously striking the cornea to be hardened. A homogeneous distribution of the electromagnetic radiation is obtained when essentially the same quantity of radiation per unit area strikes the cornea. Such a homogeneous radiation distribution is not generally achieved with a stationary point-like radiation source whose radiation strikes the spherically curved cornea, because the incidence angle of the radiation varies as a function of the position on the cornea. The invention therefore provides particular measures for homogenising the radiation distribution, so that the corneal hardening achieved by the radiation is in fact essentially homogeneous.

As a variant of the aforementioned embodiment of the invention, it is also possible to provide control instruments for the radiation distribution over the cornea so that the quantity of radiation striking the cornea per unit area can be selectively adjusted as a function of the position on the cornea, i.e. for example so that stronger hardening takes place in more peripheral regions of the cornea than in more central regions of the cornea, or vice versa, depending on the diagnosis and/or therapeutic purpose.

According to a particular configuration of the invention, an instrument is thus provided for determining properties of the cornea and/or other components of the eye. The measurements may possibly lead to varying results at different positions on the cornea, which may in turn be important for the aforementioned control of the intensity distribution of the electromagnetic radiation as a function of the position on the eye in particular embodiments of the invention.

The instrument according to the invention may be configured for hardening of the cornea by means of electromagnetic radiation, in such a way that it engages with the cornea via its shaped body which shapes the cornea. As a variant of this embodiment, the instrument with which the electromagnetic radiation is applied onto the cornea may also be configured so that it lies at a distance from the cornea. The invention also teaches various radiation sources for the electromagnetic radiation and various techniques for guiding the radiation to the place of use. Details of these can be found in the dependent patent claims and in the following description of exemplary embodiments.

According to a preferred configuration of the invention, the instrument with which the electromagnetic radiation is radiated onto the cornea is to be coupled with an operation microscope, and specifically so that the operator can observe the eye and in particular the cornea, or parts of it, during the application of the electromagnetic radiation.

According to another preferred configuration of the invention, a so-called “aligning beam” known per se is used for positioning the eye. Such a beam is occasionally also referred to as a “fixing light beam” in the literature. This makes it possible to improve the positioning of the eye with respect to the described devices and instruments. It is also possible for the devices and instruments described here to be combined with a so-called “eye-tracker”. Such “eye-trackers” are eye tracking systems which optically track possible movements of the eye and adjust other instrumentation used for surgery, for example laser beams, according to the eye's movement. According to another variant of the invention, it is also possible to support the positioning of the described devices and instruments on the eye with a spectacle frame.

The invention also teaches a method for correcting defective vision of an eye, in which deformation and hardening of the eye's cornea are carried out in combination.

Other preferred configurations of the invention will be found in the dependent patent claims and the following description of exemplary embodiments with the aid of the drawings, in which:

FIG. 1 shows a device for correcting defective vision of an eye;

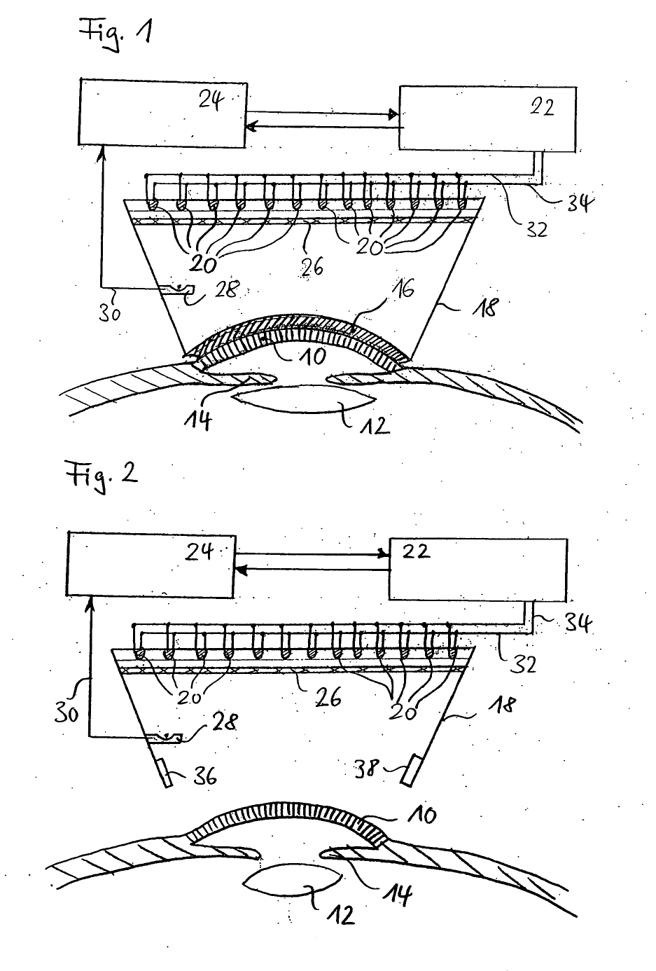

FIG. 2 shows a modified embodiment of a device for correcting defective vision of an eye;

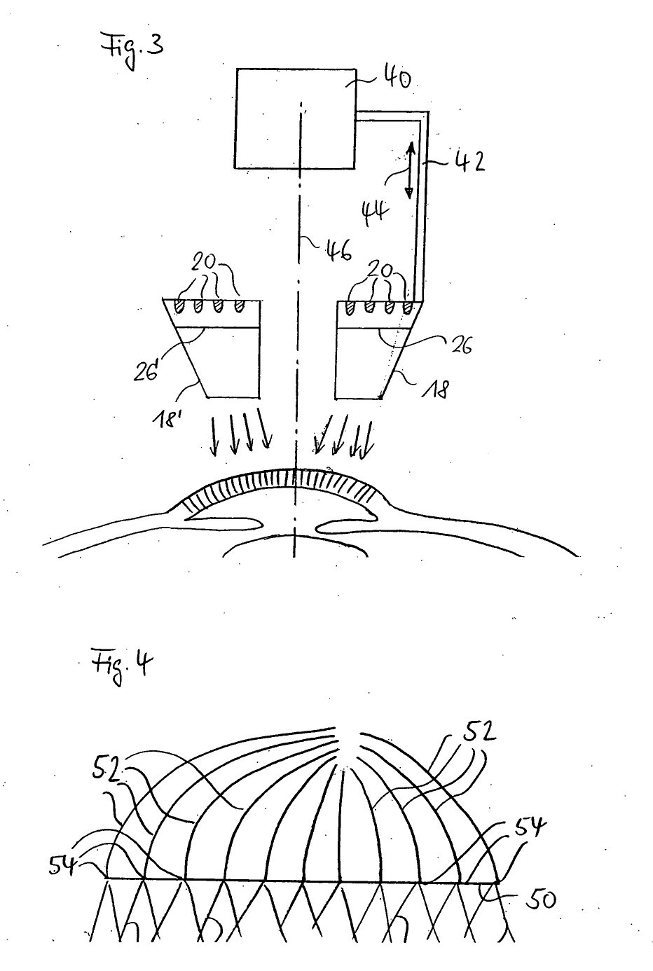

FIG. 3 shows a further exemplary embodiment of a device for correcting defective vision of an eye in combination with a microscope; and

FIG. 4 schematically shows an arrangement of a plurality of radiation sources for irradiating a cornea;

Components which correspond to one another or are functionally similar are provided with the same reference numerals in the figures.

FIG. 1 schematically shows an eye with a cornea 10, a lens 12 and an iris 14.

In the exemplary embodiment according to FIG. 1, a shaped body 16 lies directly on the cornea 10 in order to deform it in the desired way. Without the shaped body 16 (i.e. before it was pressed onto the cornea), the cornea 10 had a different shape. The shaped body 16 is firmly connected to a housing 18, which is conically shaped in the exemplary embodiment represented here in order to guide electromagnetic radiation towards the shaped body 16 and the cornea 10. The housing 18 may be mirrored on the inside for guiding the radiation.

A multiplicity of radiation sources 20 are connected to the housing 18. In the exemplary embodiment represented, the radiation sources 20 are designed as LEDs. The individual radiation sources 20 are driven in an individually adjustable way by means of a current supply 22, i.e. the quantity of radiation can be adjusted selectively, according to requirements. Either the quantity of radiation emitted by all the radiation sources 20 may be proportionally adjusted simultaneously, or individual radiation sources may be optionally adjusted selectively with respect to the quantity of radiation emitted by them, depending on their position.

A control and regulating instrument 24, which may for example be computer-controlled, is provided for controlling the quantities of radiation respectively emitted by the radiation sources 20.

A so-called “diffuser” 26, for example in the form of a scattering plate (frosted glass), a plate with a rough surface, or a transparent body with scattering centres, is arranged in the beam path of the radiation emitted by the radiation sources 20. The function of the diffuser is to distribute the radiation emitted by the radiation sources 20 as uniformly as possible so that intensity peaks are avoided.

A radiation sensor 28 detects a part of the radiation directed towards the shaped body 16 or cornea 10 by the diffuser 26, this part being representative of radiation striking the cornea 10. The measurement signal of the sensor 28 is transmitted via a line 32 to the control and regulating unit 24 for processing, so that the control and regulating unit 24 can correspondingly drive the current supply unit 22 for the individual radiation sources 20. Lines 32, 34 for the individual radiation sources 20 are schematically represented in FIG. 1, but it is preferable for each individual radiation source 20 to be selectively driveable so that different radiation intensities can be provided for the individual radiation sources.

In the exemplary embodiment according to FIG. 2, the device is modified relative to the exemplary embodiment according to FIG. 1 in so far as the instruments for generating and guiding the radiation towards the cornea are separated from the latter. To this end, the housing 18 has distance sensors 36, 38 on its ends facing the eye. The device according to FIG. 2, as well as all other devices described here for generating and guiding electromagnetic radiation, alternatively may also be used without employing a shaped body for shaping the cornea. In the exemplary embodiment according to FIG. 2 a shaped body (not shown), for example a contact lens or the like, may be applied directly onto the cornea 10.

The exemplary embodiment according to FIG. 3 shows the combination of a modified instrument for generating and guiding electromagnetic radiation in combination with a microscope 40, for example an operation microscope for eye surgery. The microscope 40 may be provided with a filter (not shown), which makes it possible for the operator to observe the eye parts of interest without problems due to the electromagnetic radiation generated by the radiation sources 20. The microscope 40 is connected to the housing 18 of the radiation sources 20 via an arm 42 and, for example, can be moved in the direction of the double arrow 44 along the optical axis 46 via a mechanism (not shown). As represented, the housing 18 with the radiation sources 20 centrally comprises a free passage for the microscope observation in the region of the optical axis 46. This opening forms an optical aperture, the central axis of which coincides with the optical axis of the microscope.

FIG. 4 schematically shows a modification of the device for generating and guiding electromagnetic radiation towards the cornea. A multiplicity of optical light guides 52 are provided according to FIG. 4, the ends 54 of which are fastened in a holding plate 50 so that the radiation cone 56 emitted by the ends emerges below the plate 50. Such an arrangement may replace the arrangement comprising the radiation sources 20 and the diffuser 26, for example in FIGS. 1, 2 and 3. The distance between the individual ends 54 of the light guides 52 and the distance from the plate 50 to the cornea can be adjusted so that the radiation cones 56 overlap enough to provide a sufficiently homogeneous radiation distribution on the cornea. Semiconductors may also be used as the light source (not shown) in this exemplary embodiment.

For example, a common radiation source (not shown) may be provided in order to feed all the light guides 52. It is also possible to drive individual light guides individually in order to permit independent adjustability of the radiation sources for at least some of the light guides. If homogeneous exposure of the cornea to electromagnetic radiation is intended to be achieved with an arrangement according to FIG. 1, 2, 3 or 4, then the spherical curvature of the cornea should be taken into account. The effect of this spherical curvature is that the radiations strike the cornea at different angles, depending on the distance from the optical axis. Differential driving of the individual light sources 20 would therefore be necessary in order to generate a fully homogeneous radiation distribution in an arrangement according to FIGS. 1 to 3.

Simple homogenisation of the radiation distribution can be achieved with an arrangement according to FIG. 4 if the plate 50 is spherically curved in the same sense as the surface of the cornea. All the cones 56 then radiate essentially radially with respect to a centre of the sphere of the corneal i.e. the axes of the individual cones are essentially perpendicular to the surface of the cornea, so that all the radiation cones 56 strike the surface in the same way and with the same angular distribution and a homogeneous radiation distribution is therefore achieved. The electronic control outlay in respect of the radiation sources is substantially simplified in this variant compared to the aforementioned variant, in which the individual radiation sources are driven so that they radiate with different intensities, depending on their position with respect to the cornea.

With the exemplary embodiments of the invention as explained with the aid of FIGS. 1 to 4, it is possible to deform and harden the cornea 10. To this end, the aforementioned photosensitiser is introduced homogeneously into the cornea in the described manner and the irradiation is carried out with suitable wavelengths, for example UVA or UV. Wavelengths in the UV range or harder radiation may currently be envisaged in particular, i.e. wavelengths approximately in a range from 300 to 400 nm. The radiation sources 20 are configured accordingly. The shaped body 16, or a contact lens used instead of it, are transparent for the radiation being employed. Overall, the entire electromagnetic radiation spectrum may in principle be envisaged, depending on the photosensitiser used and available. It is also possible to carry out corneal hardening without a photosensitiser, merely by the radiation itself.

Light-emitting diodes with different wavelengths may be used for the radiation sources 20, depending on the desired therapeutic effects. It is also possible for a light source whose radiation is guided via an optomechanical beam path (for example a so-called Köhler beam path) to be additionally used for the illumination.

According to a preferred configuration, a shaped body 16 which causes over-deformation of the cornea is used. During the contact between the shaped body or contact lens and the cornea, the latter is thus deformed more strongly than the actual deformation goal. This takes into account the fact that a certain regression, i.e. return of the cornea towards its original shape, takes place after the shaped body or contact lens is removed. The over-deformation then leads in the end to the desired shape of the cornea. The hardening with electromagnetic radiation may also be already carried out at least partially before the deformation; or else during and after the deformation. Humidifiers, anaesthetics etc. will be employed according to the diagnosis and situation.

The deformation and the hardening of the cornea with devices according to FIGS. 1 to 4 can be improved by using particular measurements on the eye.

For example, it is possible to determine the corneal thickness optically or acoustically by means which are provided in the prior art. As a function of the corneal thickness or other parameters found in this way, the process parameters can then be adjusted with a view to deforming and/or hardening the cornea, as described above. For regression-free deformation, for example, a thicker cornea will require either longer hardening times or a higher concentration of photosensitiser and/or a stronger over-deformation in the aforementioned sense.

Direct acoustic spectroscopy, to determine the biomechanical properties of the cornea during the process, is another possibility for improving the deformation and hardening with the instruments according to FIGS. 1 to 4. The said properties of the cornea, for example the degree of its hardening during the aforementioned method, can be determined by applying ultrasound (not shown) to the cornea and measuring the acoustic transmission. Control parameters for the duration of applying the electromagnetic radiation and/or its intensity may in particular be derived from this.

The prior art also includes so-called dynamic mechanical spectroscopy for determining biomechanical properties of the cornea. This technique may also be used in combination with the disclosed devices and methods, in order to optimise the process parameters.

So-called fluorescence analysis is likewise known per se, and this is particularly suitable for monitoring the intensity of the applied radiation as well as its effects, and in turn deriving control parameters for the irradiation from the values which are found, i.e. for example attenuating the radiation in particular situations in order to avoid undesired effects.

It is also possible for convocal microscopy, which is known per se, to be used together with the disclosed devices in order to assess tissue effects which may possibly occur, in order to avoid undesired interference. Provision may also be made to determine the internal pressure of the eye during use of the device, possibly in order to derive control quantities from this for hardening the cornea. Similar considerations apply to the use of optical spectroscopy methods which are known per se for tissue characterisation, or even methods which permit tissue characterisation by means of acousto-optical spectroscopy.

The current supply of the aforementioned devices and instruments may optionally be carried out using a battery, an accumulator or using a power supply unit. It is also possible to use an electromechanically displaceable patient support or a corresponding chair for positioning the patient's eye.

The aforementioned devices and instruments may be combined with a surgical laser system for refractive corrections on the eye. This may, for example, involve a LASIK system which is well known per se to the person skilled in the art. By means of such a combination of the devices according to the invention with a known LASIK system, for example, it is possible to carry out cross-linking of the cornea in a LASIK operation in which the cornea is reshaped, for example after or during the LASIK operation. It is thereby possible to extend the corrective range in the LASIK method.

1. Device for correcting defective vision or corneal disease of an eye, characterised by the combination of
an instrument (16) for deforming the cornea of the eye with
an instrument (18, 20) for hardening the cornea
at least one radiation source (20) for irradiated the cornea,
one or more radiation sources (20) in the instrument being arranged so that the radiation emitted by them strikes the cornea homogeneously.

2. Device according to claim 1, characterised in that the instrument (16) for deforming the cornea comprises a shaped body which can be placed on the eye.

3. Device according to claim 1, characterised in that the instrument is configured so that it can be brought in contact with the cornea for proper use.

4. Device according to claim 1, characterised in that the instrument is configured so that it lies at a predetermined distance from the cornea for proper use.

5. Device according to claim 1, characterised in that light-emitting diodes are provided as the radiation source.

6. Device according to claim 1, characterised by a radiation source with optical waveguides (52).

7. Device according to claim 1, having a conical body (18) for guiding the radiation.

8. Device according to claim 1, having a radiation sensor (28) for detecting a part of the radiation emitted by the radiation source or radiation sources.

9. Device according to claim 1, characterised by a control or regulating instrument (24) which can control or regulate the radiation.

10. Device according to claim 1, characterised by a device (36, 38) for measuring the distance between a component of the device and the cornea.

11. Device according to claim 1, characterised in that the device comprises a plurality of radiation sources (20) which are arranged so that their radiation cones (56) allow homogeneous illumination of a cornea by overlapping.

12. Device according to claim 1, having a device (22) for driving individual radiation sources.

13. Device according to claim 1, having means for determining properties of the cornea.

14. Operation microscope combined with a device according to claim 1.

15. Device having a surgical laser system for refractive corrections of the cornea, in combination with a device according to claim 1.

16. Device according to claim 2, characterised in that the instrument is configured so that it can be brought in contact with the cornea for proper use.
